(19) United States
(12) Patent Application Publication
(10) Pub. No.: US 2020/0289645 A1
(43) Pub. Date: Sep. 17, 2020

(54) TREATMENTS AND METHODS FOR CONTROLLING HYPERTENSION

(71) Applicant: Georgetown University, Washington, DC (US)

(72) Inventor: Anton Wellstein, Washington, DC (US)

(73) Assignee: Georgetown University, Washington, DC (US)

(21) Appl. No.: 16/759,354

(22) PCT Filed: Nov. 19, 2018
(86) PCT No.: PCT/US18/61834

§ 371 Date: Apr. 26, 2020

Related U.S. Application Data
(60) Provisional application No. 62/588,675, filed on Nov. 20, 2017.

(51) Int. Cl. A61K 39/395; A61K 38/17; A61K 45/06; A61K 47/68

(52) U.S. Cl. CPC A61K 39/3955; A61K 47/6817; A61K 45/06; A61K 38/179

(57) ABSTRACT

The present invention relates to methods of treating hypertension in a subject in need of treatment thereof, with the methods comprising administering a pharmaceutically effective amount of an angiotensin II inhibitor and a pharmaceutically effective amount of a receptor tyrosine kinase inhibitor to the subject.

### STATEMENT REGARDING FEDERALLY SPONSORED RESEARCH OR DEVELOPMENT

This invention was made with Government support under grant no. P01 HL068686 and R01 CA71508 awarded by National Institutes Health. The government has certain rights in the invention.

### REFERENCE TO SEQUENCE LISTING

N/A

### BACKGROUND OF THE INVENTION

### Field of the Invention

The present invention relates to methods of treating hypertension in a subject in need of treatment thereof, with the methods comprising administering a pharmaceutically effective amount of an angiotensin II inhibitor and a pharmaceutically effective amount of a receptor tyrosine kinase inhibitor to the subject.

### Background of the Invention

The family of fibroblast growth factors (FGFs) encompasses eighteen FGF receptor ligands and seven distinct receptor proteins with a wide expression range. They have distinct roles in embryonic development, in adult organ homeostasis and vascular adaptation as well as in a wide range of diseases. Genome-wide association studies in hypertensive populations have shown a potential role of molecules in the FGF pathway. Polymorphisms in the FGF5 locus have been associated with blood pressure regulation and hypertension in large populations of European and of Japanese ancestry. Variations in the FGF1 locus have been correlated with familial hypertension and with the upregulation of FGF1 expression in kidneys. In addition, genomic analysis has revealed that a polymorphism in the FGFBP1 locus was associated with familial hypertension and hypertensive subjects showed increased expression of BP1 mRNA and protein in renal tissues. Studies in hypertensive rats have corroborated a contribution of the FGFBP1 genomic locus to glomerular damage and to hypertension.

Secreted FGF binding proteins (BPs or FGFBPs) shuttle paracrine-acting FGFs from their extracellular matrix storage sites to their receptors and thus enhance their signaling. BP1 (originally named HBp17 13 and FGFBP) is the best characterized of the three known members of the family and interacts via its C-terminus with FGF1, 2, 7, 10 and 22 in a reversible, noncovalent manner. Depletion of endogenous BP1 reduces FGF2 release and blunts tumor growth and angiogenesis of human cancer cells and resulted in distinct developmental defects during chick embryogenesis. On the other hand, BP1 is upregulated in angioproliferative Kaposi Sarcoma, contributes to an angiogenic phenotype of cultured endothelial cells and controls angiogenesis and wound healing in adult mice.

### SUMMARY OF THE INVENTION

The present invention relates to methods of treating hypertension in a subject in need of treatment thereof, with the methods comprising administering a pharmaceutically effective amount of an angiotensin II inhibitor and a pharmaceutically effective amount of a receptor tyrosine kinase inhibitor to the subject.

### BRIEF DESCRIPTION OF THE DRAWINGS

FIG. 1 depicts conditional BP1 transgene expression in kidneys in vivo. 1A, BP1 mRNA expression in kidneys of BP1 OFF and ON animals by quantitative RT-PCR. Expression was normalized to endogenous beta-actin mRNA (means±SEM; n=8 and 10 animals per group). *, P<0.05, BP1 ON versus OFF. 1B, Detection of BP1 protein by immunohistochemistry in kidneys from BP1 OFF and ON mice (total n=3). Size bar: 50 μm.

FIG. 2 depicts the effect of conditional expression of the BP1 transgene and of inhibitors on mean arterial pressure (MAP) and heart rate (HR). 2A, 2B, Circadian changes in MAP (A) and HR (B) by telemetry of conscious mice. Averages for 10 days before (BP1 OFF, open symbols) and 10 days after the induction of the BP1 transgene (BP1 ON, closed symbols) relative to the 24 hour clock time. Dark and light periods are indicated. Mean±SEM values (for n=10 time points) from one representative animal (total n=6). P<0.0001 by non-linear regression analysis and ANOVA. 2C, Effect of induction of BP1 transgene expression on night time MAP. Gene expression was induced by switching from a tetracycline containing to a regular chow (+DOX to −DOX; open and filled circles). Non-transgenic littermates (non-TG; triangles) were subjected to the same diet schedule. Mean±SEM of night time MAP; n=6 per group. ANOVA analysis of BP1 ON versus OFF or versus control group. ***, P<0.0001. 2D, 2E, Effect of treatment with candesartan (D) or tempol (E) on night time MAP of animals with the BP1 transgene OFF or ON. Mean±SEM; n=5 per group. *, P<0.05; **, P<0.01; ***, P<0.0001 by ANOVA.

FIG. 3 depicts the effect of the induction of BP1 expression on cremaster arteriole contractility in vivo. 3A, Representative images of a single arteriole in a live animal at baseline, and after constriction or dilation by superfusion with AngII or acetylcholine respectively. The vessel diameter is recorded by intravital microphotography. 3B, Dose-response of AngII on arteriole diameter in mice with BP1 OFF (open symbols) or ON (for 48 hours; closed symbols). The FGFR kinase inhibitor PD173074 (PD) was administered intraperitoneally to a subset of animals (red symbols). Mean±SEM, n=4 to 7 animals/group. pEC50 values of 6.64+0.15 (=230 nM; BP1 OFF) and 7.92+0.13 (=12 nM; BP1 ON) were calculated from non-linear regression analysis. ***, P<0.0001 BP1 OFF vs. BP1 ON and BP1 ON+PD vs. BP1 ON. 3C, Dose response of Phenylephrine (PE) on cremaster arteriole diameter in mice with BP1 OFF (open symbols) or ON (for 48 hours; closed symbols). Mean±SEM, n=4 animals/group.

FIG. 4 depicts the effect of BP1 on isolated renal afferent arteriole contractility. 4A, Isolated perfused renal afferent arteriole mounted on a perfusion pipette with diameter recorded. The internal diameter is 10 to 11 micrometers under control conditions (=100%). 4B to 4D, Impact of BP1 induction and/or FGF2 knockout (FGF2−/−) on angiotensin II (AngII) or norepinephine (NE) contractility. 4B, Effect of conditional BP1 expression (BP1 ON/OFF). Expression of BP1 increases the AngII effect (open versus filled black circles). Addition of the FGFR kinase inhibitor PD173074 (100 nM) inhibits the effect of AngII. The effect of AngII in FGF2−/− mice is shown for comparison (see panel C). 4C, Effect of AngII in afferent arterioles from FGF2−/− mice. Add-back of FGF2 plus BP1 proteins (20 ng/ml for 30 min) restores the AngII effect. BP1 alone or FGF2 alone are shown for comparison. 4D, Effect of NE in afferent arterioles from wt and FGF2−/− mice. Contractility induced by NE is not affected in FGF2−/− mice.

FIG. 5 depicts the signal transduction changes in kidneys after the induction of BP1. 5A, Gene expression data were subjected to an analysis for Upstream Regulators using the Ingenuity platform. z-scores and p-values (−log) are shown. Relevant data points are labeled. 5B, Hallmark pathways from a Gene Set Enrichment Analysis of the gene expression data. NES, normalized enrichment score; q, false discovery adjusted p-value. 5C, Detection of phospho-MKK4 or phospho-p38 or phospho-JNK by immunohistochemistry of kidneys from BP1 OFF and ON mice (total n=3). Size bar: 50 μm. 5D, Phospho-p38 Western blot analysis of kidney extracts from BP1 OFF and ON animals after immunoprecipitation for phospho-p38. Total p38 protein is shown for comparison.

FIG. 6 depicts the integration of signaling changes after the induction of BP1. The crosstalk between FGFR and AngII/GPCR signaling based on the Upstream Regulator, pathway and protein analyses. Signaling proteins identified by tissue staining (pMKK4, pJNK, pp38) or by mass spectrometry analysis of phospho-tyrosine protein complexes isolated from tissues (CIT, MKK4, PAK2, PTPN12) are underlined. Inhibitors used in the experiments are also shown.

### DETAILED DESCRIPTION OF THE INVENTION

Activation of G-protein coupled receptors (GPCRs) and receptor tyrosine kinases such as the FGFR initiate converging signaling cascades in cells to elicit a phenotypic response. Earlier studies in rat aortic smooth muscle cells showed an increase of AngII-stimulated Ca2+ release after treatment with FGF2 and a blockade of the increase after inhibition of the MAP kinase pathway suggesting a crosstalk that could be relevant for the initiation of hypertension. In addition, FGF2 was found to be essential for mediating AngII-induced cardiomyopathy that utilized the JNK and p38 MAP kinase pathways. Moreover, endogenous FGF2 has been implicated in pulmonary hypertension and cardiac hypertrophy, both of which are conditions associated with increased vascular resistance.

Some mechanistic insight into the crosstalk between pathways that control vascular tone and angiogenic signals emerged from inhibitors of VEGF-driven tumor angiogenesis used to treat cancers. VEGF pathway activity is restricted to endothelial cells and the major side effect of systemic therapy with VEGF pathway inhibitors is hypertension due to the reduction of constitutive eNOS. FGFs also act on endothelial cells and their effects overlap with VEGF. Indeed, intravascular administration of exogenous FGF1 or FGF2 lowers blood pressure in experimental animals and can correct hypertension due to preferential targeting of endothelial signaling due to this route of administration. Because endogenous FGFs also act on vascular smooth muscle cells, this balance of their endothelial activity explains the apparent paradox that FGF2-deficient mice are hypotensive despite the hypotensive effect of intravenously administered FGF2. Interestingly AngII also shows distinct activity on blood pressure that depends on the cell type stimulated. Typically, AngII will cause vasoconstriction and a rise in blood pressure. AngII, however, reduced blood pressure in animals that only expressed endothelial AngII receptors due to the vasodilatory effects of the endothelial stimulus.

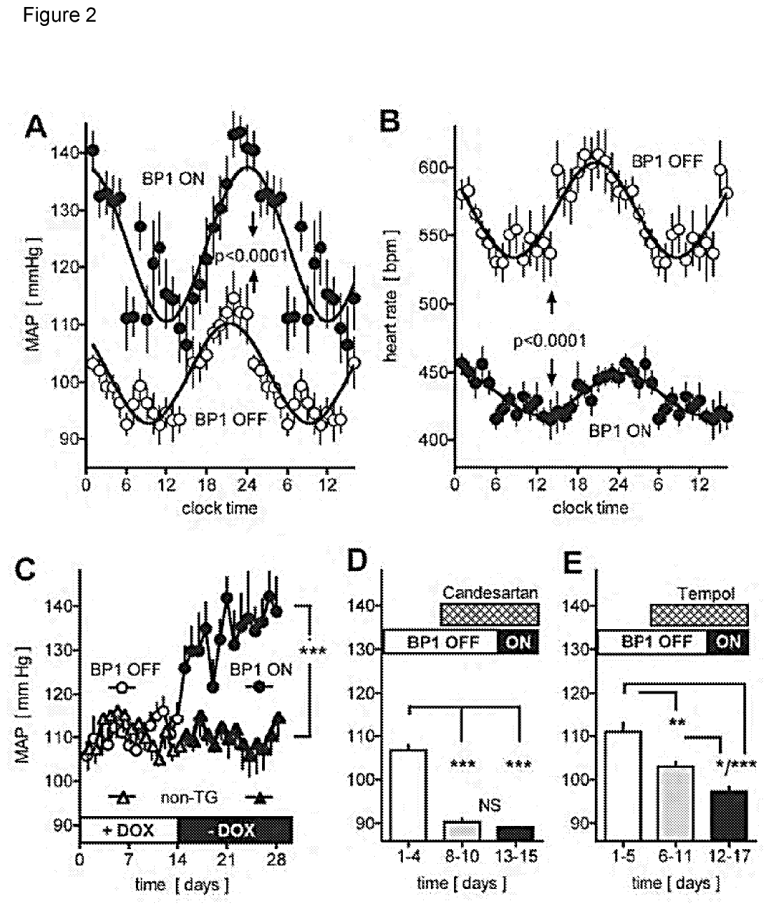

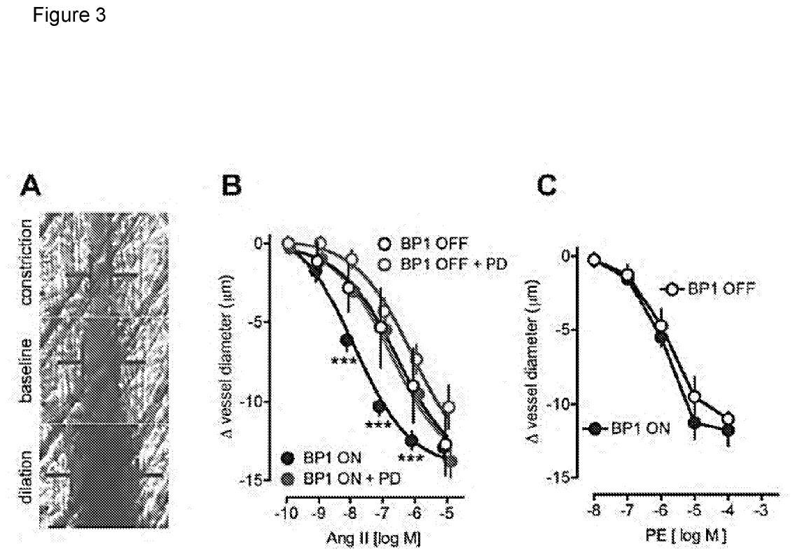

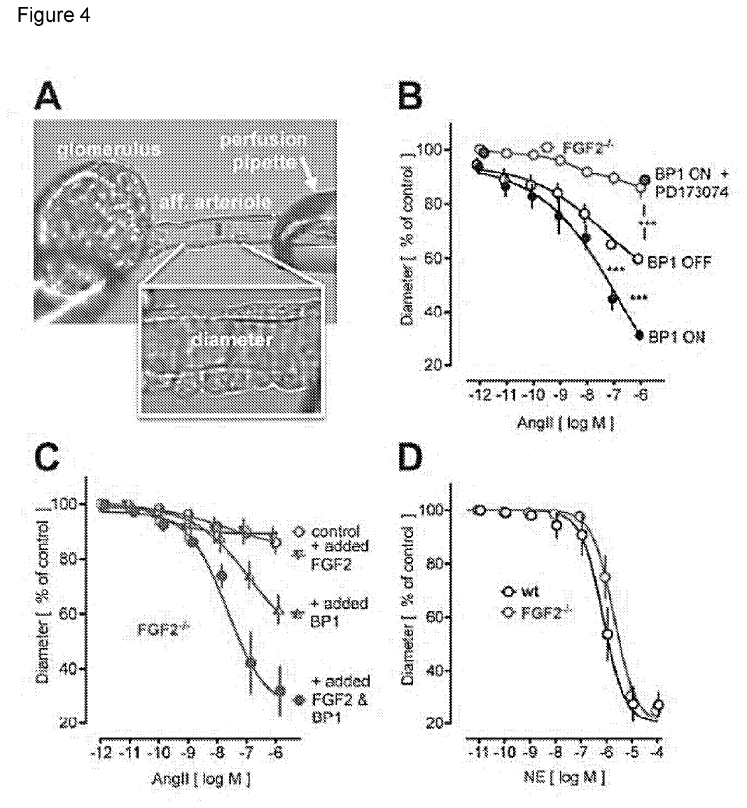

The predominant effect of FGF pathway activation by the induction of BP1 expression is to increase blood pressure by sensitizing resistance vessels to AngII (FIGS. 3 and 4). This sensitization to AngII vasoconstriction was reversed by an FGFR kinase inhibitor, supporting the notion that increased BP1 expression activates the FGF pathway and thus increases vessel sensitivity for AngII. The increased baseline sensitivity of resistance vessels was reflected in increased blood pressure that is dependent on AngII receptor signaling as evidenced by the inhibitory effects of the receptor antagonist Candesartan (FIG. 2D).

When evaluating the G-protein coupled receptors (GPCR) and FGFR crosstalk further, a qualitative difference was found between AngII and alpha-adrenoceptor sensitization of arteriolar contractility, after FGF pathway activation. This difference is likely due to the distinct downstream effectors of these GPCRs. Previous studies have shown that chronic AngII infusion increased vascular superoxide, which enhanced the pressor response and increased arteriole constriction by AngII, but not by norepinephrine. Thus, distinct intracellular effectors can modulate the crosstalk of FGFR and AngII.

The present invention relates to methods of treating hypertension in a subject in need of treatment thereof, with the methods comprising administering a pharmaceutically effective amount of an angiotensin II inhibitor and a pharmaceutically effective amount of a receptor tyrosine kinase inhibitor to the subject.

Fibroblast Growth Factors (FGFs) participate in organ development and tissue maintenance as well as the control of vascular function. The paracrine-acting FGFs are stored in the extracellular matrix and their release is controlled by a secreted FGF-binding protein (FGF-BP, FGFBP1, BP1) that modulates FGF receptor (FGFR) signaling. A genetic polymorphism in the human FGFBP1 gene is associated with higher gene expression and an increased risk of familial hypertension.

Induction of BP1 expression in adult animals leads to a sustained rise in mean arterial pressure by >30 mm Hg. The hypertensive effect of BP1 expression was prevented by administration of candesartan, an angiotensin II (AngII) receptor antagonist, or by tempol, an inhibitor of reactive oxygen species. The in vivo expression of BP1 sensitizes peripheral resistance vessels to AngII constriction by 20-fold but does not alter adrenergic vasoconstriction. FGFR kinase inhibition reverses the sensitization to AngII.

In addition, constriction of isolated renal afferent arterioles by AngII was enhanced after BP1 expression and blocked by FGFR kinase inhibition. Furthermore, AngII-mediated constriction of renal afferent arterioles was abolished in FGF2−/− knockout mice, but was restored by add back of FGF2 plus BP1 proteins. In contrast to AngII, adrenergic constriction was not affected in the FGF2−/− model. Proteomics and gene expression analysis of kidney tissues after BP1 induction showed that MAP kinase signaling via MKK4, p38 and JNK integrates the crosstalk of the FGFR and AngII pathways and thus impacts vascular tone and blood pressure.

Angiotensin II (AngII) is a well-characterized hormone that is eight amino acids long and is known to be involved in regulating blood pressure. As used herein angiotensin II inhibitors are well-known in the art and include compounds and methods that prevent or diminish the production of AngII, as well as those compounds or methods designed to prevent or diminish the activity of AngII. In one embodiment, the AngII inhibitor is an inhibitor of angiotensin converting enzyme (ACE). Examples of ACE inhibitors that can be used in the methods of the present invention include but are not limited to benazepril, captopril, enalapril fosinopril, Lisinopril, moexipril, perindopril, quinapril, ramipril and trandolapril.

In another embodiment, the AngII inhibitor is an AngII receptor antagonist. Examples of AngII receptor antagonists include but are not limited to Olmesartan, Telmisartan, Losartan, Irbesartan, Valsartan, Candesartan, Eprosartan, Azilsartan, Losartan/hydrochlorothiazide, Amlodipine/valsartan, Telmisartan/hydrochlorothiazide and Valsartan/hydrochlorothiazide.

The methods of the present invention comprise administering an AngII inhibitor with an inhibitor of a receptor tyrosine kinase (RTK). As used herein, a receptor tyrosine kinase inhibitor (RTKi) includes those compounds and methods that are designed to inhibit the activity or function of a receptor tyrosine kinase. RTKs are well-known in the art and are cell surface receptors that dimerize upon ligand binding, which, in turn, activates the tyrosine kinase activity.

In one embodiment, the RTKi is an inhibitor of the activity of fibroblast growth factor receptor 1 (FGFR1). In another embodiment, the RTKi is an inhibitor of the activity of fibroblast growth factor receptor 2 (FGFR2). In another embodiment, the RTKi is an inhibitor of the activity of fibroblast growth factor receptor 3 (FGFR3). In yet another embodiment, the RTKi is an inhibitor of the activity of fibroblast growth factor receptor 4 (FGFR4).

In even more specific embodiments, the RTKi is a tyrosine kinase inhibitor. As used herein a tyrosine kinase inhibitor is a molecule that inhibits the activity or function of a tyrosine kinase, including receptor tyrosine kinases and non-receptor (cytoplasmic) tyrosine kinases. Thus the phrase “tyrosine kinase” as used herein includes receptor tyrosine kinases and non-receptor (cytoplasmic) tyrosine kinases. Accordingly, a tyrosine kinase inhibitor (TKi) can include a receptor tyrosine kinase inhibitor (RTKi) or a non-receptor (cytoplasmic) tyrosine kinase inhibitor (CTKi).

Examples of RTKi's include but are not limited to PD173074 (CAS No. 219580-11-7), AZD4547 (CAS No. 1035270-39-3), BGJ398 (CAS No. 872511-34-7), AP24534 (CAS No. 943319-70-8), BIBF1120 (CAS No. 656247-17-5), JNJ-42756493 (CAS No. 1346242-81-6), TKI-258 (CAS No. 405169-16-6), PHA-739358 (CAS No. 827318-97-8), BMS-540215 (CAS No. 649735-46-6), TKI-258 dilactic acid (CAS No. 852433-84-2), MK-2461 (CAS No. 917879-39-1), BMS-582664 (CAS No. 649735-63-7), SSR128129E (CAS No. 848318-25-2), PRN1371 (CAS No. 1802929-43-6), PD166866 (CAS No. 192705-79-6), BLU554 (CAS No. 1707289-21-1), S49076 (CAS No. 1265965-22-7), SU5402 (CAS No. 215543-92-3), BLU9931 (CAS No. 1538604-68-0), FIN-2 (CAS No. 1633044-56-0), TKI-258 lactate (CAS No. 915769-50-5), CH5183284 (CAS No. 1265229-25-1) or LY2874455 (CAS No. 1254473-64-7). As one is well aware, the CAS (chemical abstracts service) number assigned to each molecule is a unique identifier for each compound.

In other specific embodiments, the RTKi is a ligand trap. As used herein, a ligand trap is generally a protein, or perhaps some other type of molecule, that is designed to bind to a ligand and thereby prevent the ligand from binding to its cognate receptor. As used here, a ligand trap need not bind the target ligand with the same affinity as that of the receptor, so long as the binding of the ligand to its cognate receptor is at least hampered or diminished. In one specific embodiment, the ligand trap that is administered to the subject it a ligand trap that traps at least one of the members of the FGF family of proteins. In specific embodiments, the ligand trap is a molecule that binds at least FGF2. In one specific embodiment, the RTKi that is administered to the subject is the FP-1039 ligand trap (GSK3052230) that is disclosed and characterized in Harding T., et al., Sci. Transl. Med. 5:178ra39 (2013), which is incorporated by reference.

In still other specific embodiments, the RTKi is an antibody specific for the receptor tyrosine kinase. The RTK-specific antibodies can be monoclonal or polyclonal and may be human or humanized antibodies. In one embodiment, the RTK-specific antibody is GP369, which is described in Bai, A., et al., Cancer Res., 70(19):7630-7639 (2010), which is incorporated by reference. In another embodiment, the RTK-specific antibody is BAY1187982, which is described in Sommer, A., et al., Cancer Res., 76(21):6331-6339 (2016). In yet another embodiment, the RTK-specific antibody is MFGR1877S (also known as RG7444), which is described in ODonnell, P., et al., Eur. J. Cancer, 48(6):191-192 (2012), which is incorporated by reference.

The term hypertension is used as it is in the art and includes primary hypertension (no identifiable cause) and secondary hypertension (caused by underlying condition). In one embodiment, the subject is diagnosed with primary hypertension prior to the administration of the AngII inhibitor and the RTKi. In another embodiment, the subject is diagnosed with secondary hypertension prior to the administration of the AngII inhibitor and the RTKi. The diagnosis of the hypertension may depend on the subject's age, race, family history, weight status, level of activity, tobacco use and dietary factors. Moreover, the hypertension in the subject may asymptomatic or may present symptoms such as but not limited to headaches, shortness of breath and even nosebleeds.

As used herein, “administering,” and “administer” are used to mean introducing one or more compounds into a subject. When administration is for the purpose of treatment, the composition is provided at, or after the onset of, a symptom or condition in need of treatment. The therapeutic administration of this composition serves to attenuate any symptom, or prevent additional symptoms from arising. When administration is for the purposes of preventing a condition from arising (“prophylactic administration”), the composition is provided in advance of any visible or detectable symptom. The prophylactic administration of the composition serves to attenuate subsequently arising symptoms or prevent symptoms from arising altogether. The route of administration of the composition includes, but is not limited to, topical, transdermal, intranasal, vaginal, rectal, oral, subcutaneous intravenous, intraarterial, intramuscular, intraosseous, intraperitoneal, epidural and intrathecal as previously disclosed herein.

Furthermore, the methods include coadministering one or more compounds to the subject. The term “coadminister” indicates that each of at least two substances is administered during a time frame wherein the respective periods of biological activity or effects of each of the substances overlap. Thus the term includes sequential as well as coextensive administration of the AngII inhibitor and RTKi with one another. And similar to administering the single substances, coadministration of more than one substance can be for therapeutic and/or prophylactic purposes. If more than one substance is coadministered, the routes of administration of the two or more substances need not be the same.

The proper dosages depend on various factors such as the type of disorder being treated, the particular compositions being used and the size and physiological condition of the patient. Therapeutically effective doses for the compounds to be administered can be estimated initially from cell culture and animal models. For example, a dose can be formulated in animal models to achieve a circulating concentration range that initially takes into account the IC₅₀ as determined in cell culture assays. The animal model data can be used to more accurately determine useful doses in humans.

### EXAMPLES

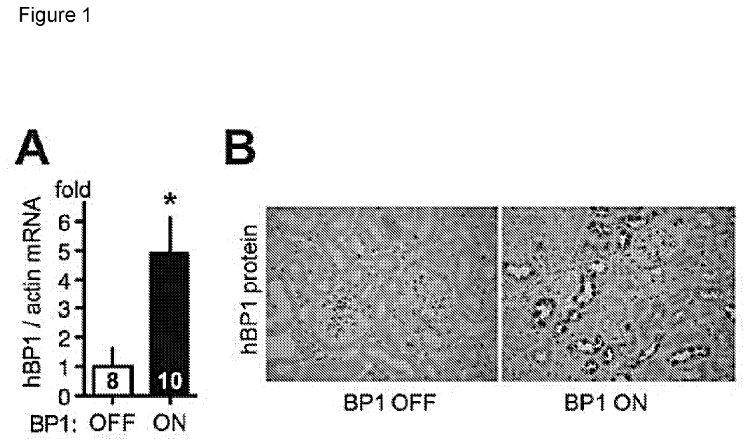

BP1 transgene expression results in embryonic lethality due to vascular leakage. Thus, a conditional transgenic mouse model was established in which BP1 transgene expression is repressed by tetracycline (“OFF”) and induced by switching animals to a regular diet (“ON”) thus avoiding the negative impact of embryonic gene expression. In vivo regulation of conditional BP1 mRNA and protein expression in kidneys of transgenic animals was confirmed by quantitative RT-PCR (qRT-PCR) and staining of formalin-fixed, paraffin-embedded kidneys from BP1 OFF and ON transgenic animals (FIG. 1A,B). Inducible BP1 mRNA and protein expression was also confirmed in heart and lung tissues by qRT-PCR and by Western blot analysis that showed the BP1 protein migrating at the predicted mass of 34 kDa after induction of expression. Overall, BP1 mRNA was inducible by 3- to 5-fold.

To test the effect of conditional expression of BP1, mean arterial pressure (MAP) was monitored by telemetry in conscious transgenic mice. Under control conditions (BP1 OFF) there was a circadian rhythm of MAP that varied by 20 mm Hg between periods of activity (night time) and rest (day time). A sinus wave function described the day/night variation of the data (FIG. 2A). Analysis of a 10 day period before and after induction of the BP1 transgene in the same animals showed a rise of MAP during the activity phase by >30 mm Hg (FIG. 2C) and an almost doubling of the MAP changes between activity and rest periods (FIG. 2A). This overall increase in MAP coincided with a significant decrease in the heart rate and a dampened amplitude of circadian regulation (FIG. 2B). The increase of blood pressure occurred within two days of a switch to the tetracycline-free, regular diet in parallel with the induction of the BP1 transgene (FIG. 2C). Switching non-transgenic littermates from the tetracycline-containing to the regular diet did not change MAP (FIG. 2C).

The increase in MAP and decrease in heart rate after induction of BP1 expression (FIG. 2A,B) was reminiscent of an angiotensin-like vasopressor effect that depends on reactive oxygen species (ROS) signaling. To assess whether endogenous Angiotensin II (AngII) receptor signaling and ROS contribute to BP1-mediated hypertension, mice were treated with the receptor antagonist Candesartan or the redox-cycling antioxidant nitroxide tempol. Candesartan reduced basal MAP (FIG. 2D) and prevented the increase of MAP after BP1 induction (FIG. 2D). Tempol also reduced basal MAP (FIG. 2E) and prevented the increase in MAP after BP1 induction (FIG. 2E).

To evaluate whether conditional BP1 expression sensitized resistance vessels in vivo, cremaster arterioles were exposed in anesthetized mice and superfused locally with vasoconstrictor or -dilator ligands. Representative intravital microscopic images of an arteriole at baseline, with AngII (constriction) or acetylcholine (dilation) superfusion is shown in FIG. 3A. There was a 20-fold sensitization of the AngII vasoconstrictor response after the induction of BP1 gene expression (EC50=230 nM versus 12 nM; FIG. 3B; p<0.0001 BP1 OFF versus ON). This sensitization was prevented by pretreatment of animals with the FGFR kinase inhibitor PD173074 23 (FIG. 3B). Control animals (BP1 OFF) showed only a small and insignificant effect after the FGFR kinase inhibitor (FIG. 3B).

Adrenergic receptor activation in resistance arterioles, unlike AngII, does not induce ROS generation and induces vasoconstriction that is not enhanced by oxidative stress. Thus, phenylephrine (PE) was selected as a ligand that activates alphal-adrenergic receptors. The extent of vasoconstriction by PE was similar to AngII (FIG. 3B). But, unlike AngII, BP1 expression did not sensitize vessels to PE (FIG. 3C). Neither the EC50 (2 μM) nor the maximal effect of PE were different between the BP1 ON and OFF groups (FIG. 3C). Thus, conditional expression of BP1 sensitizes arterioles to AngII in vivo. This sensitization was dependent on FGFR signaling.

Isolated vessels provide an approach for the analysis of vascular function that is not affected by systemic cardiovascular regulation in the intact animal. BP1 up-regulation related to human hypertension was found in the kidneys that are key organs in systemic blood pressure regulation. Thus, the impact of BP1 expression was evaluated in isolated renal afferent arterioles that are the major renal resistance vessels. FIG. 4A shows the experimental set-up with a glomerulus and an afferent arteriole mounted onto a perfusion pipette. Addition of AngII to this preparation leads to a concentration-dependent constriction. Afferent arterioles isolated from mice after conditional BP1 transgene expression showed a significantly enhanced effect of AngII (FIG. 4B). Pretreatment of the vessels with the FGFR kinase inhibitor PD173074 inhibited the effect of AngII (FIG. 4B) to a level similar to the response in arterioles from FGF2−/− mice. Thus, the AngII contractile effect depends on FGFR signaling in isolated vessels as well as in arterioles in vivo (FIG. 3B).

Kidneys contain amongst the highest concentrations of FGF2 protein that is immobilized in the extracellular matrix and can be released by BP1. To assess a contribution by FGF2, the efficacy of AngII was investigated and the crosstalk with BP1 in renal afferent arterioles from FGF2−/− mice. FGF2−/− mice showed a reduced vascular tone and lower blood pressure. It was confirmed by telemetric measurements that mean arterial pressure (MAP) in FGF2−/− mice is reduced significantly by 15 mm Hg relative to wild-type (wt) mice. Also, the effect of exogenously administered AngII on MAP was reduced.

Consistent with the blood pressure effect, AngII failed to induce a contraction in renal afferent arterioles isolated from the FGF2−/− mice (FIGS. 4B and C). The blockade of AngII effects in vessels from wt mice by an FGFR kinase inhibitor (FIG. 4B) corroborates the crucial role of FGF signaling for AngII efficacy. It is noteworthy that vessels from FGF2−/− mice still contracted in response to other ligands. For example, the effect of norepinephrine (NE) was indistinguishable between FGF2−/− and wt controls (FIG. 4D) indicating a selective crosstalk between AngII and FGF signaling. Renal AngII receptor 1 (AT1R) mRNA was not changed upon induction of BP1 expression or in FGF2−/− mice.

Because FGF2 and BP1 are extracellularly acting proteins, recombinant proteins were added to renal afferent arterioles from FGF2−/− mice to assess if this would rescue AngII vasoconstriction. Whilst FGF2 alone did not impact the AngII response, most likely due to its capture by the extracellular matrix, the combination of FGF2 and BP1 restored a full contractile response of AngII (FIG. 4C).

An unbiased gene expression analysis by cDNA array was undertaken in kidneys before and two days after induction of BP1, i.e., after the initial rise in blood pressure (FIG. 2C). Upstream regulators were identified using the Ingenuity platform and Gene Set Enrichment Analysis (GSEA) to detect expressions patterns associated with signaling pathways.

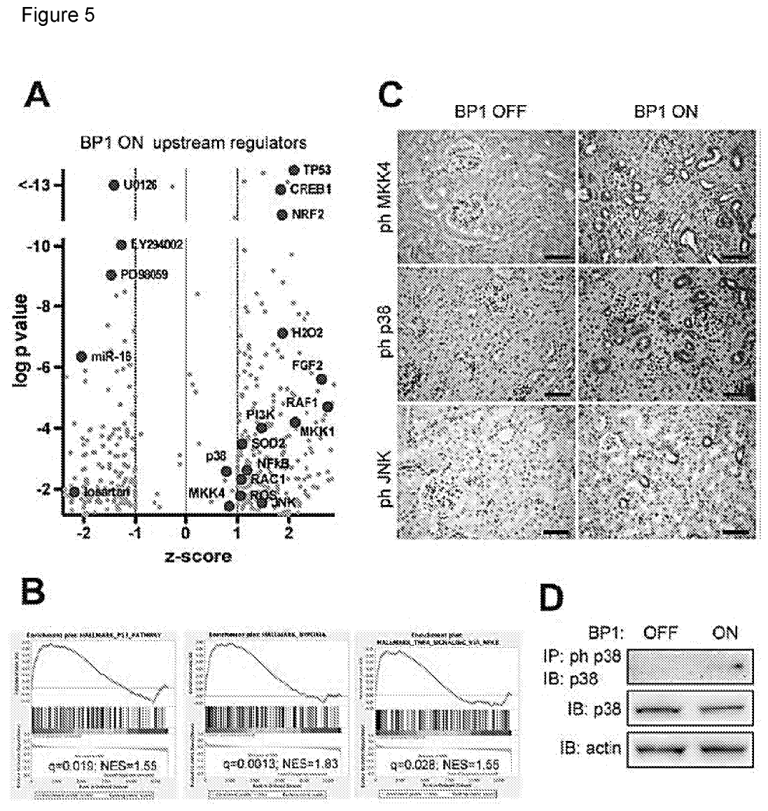

The upstream regulator analysis (FIG. 5A) showed positive z-scores>+1 and p-values<10⁻¹⁰ for the gene sets controlled by the transcriptional regulators TP53 (Tumor Suppressor P53), NRF2 (NFE2L2, nuclear factor erythroid-derived 2-like 2) and CREB1 (cAMP responsive element binding protein 1). TP53 modulates cellular stress response genes, NRF2 regulates anti-oxidant genes and ChIPseq has shown an overlap of the target genes of NRF2 and of CREB1. This analysis corroborates the contribution of ROS signaling after BP1 induction that was shown to contribute to the effect in functional studies with the antioxidant tempol (FIG. 2E). FGF2 was also identified as one of the upstream regulators. The loss of the AngII contractile response in renal afferent arterioles from FGF2−/− mice (FIG. 4C) and the inhibition of AngII-mediated vascular contractility by an FGFR kinase inhibitor (FIG. 3B; 4B) supports a regulatory role of FGF2.

Negative z-scores<−1 in the upstream regulator analysis were related to drugs that can inhibit effects of BP1 expression, i.e., AT1R (losartan), MKK (U0126; PD98059), PI3K (LY294002). In addition, microRNA-16 (miR-16) was indicated as a significant (p<10−6) upstream regulator with a negative z-score<−2. miR-16 controls endothelial cell response to growth factors in vivo and thus provides a negative feed-back loop for growth factor signaling.

The gene set enrichment analysis showed significantly impacted “hallmark” pathways that are exemplified in FIG. 5B. P53 was identified as a hallmark pathway which matches with the identification of P53 as an upstream regulator. The “hypoxia hallmark” pathway can be integrated with the NRF2 upstream regulator and “TNF-alpha pathway signaling via NFκB” matches with NFκB as an upstream regulator.

It was hypothesized that analysis of changes in protein phosphorylation and formation of signaling complexes could provide additional insight into altered pathways in BP1 ON versus OFF mice. Kidney proteins were extracted with mild detergent to maintain protein/protein interactions, and protein complexes captured with an immobilized anti-phospho-tyrosine (pY) monoclonal antibody. As established previously, changes in signal complexes can thus be revealed if one of the protein partners in the complexes is tyrosine phosphorylated (pY). 2D gel electrophoresis was used to separate the pY containing protein complexes.

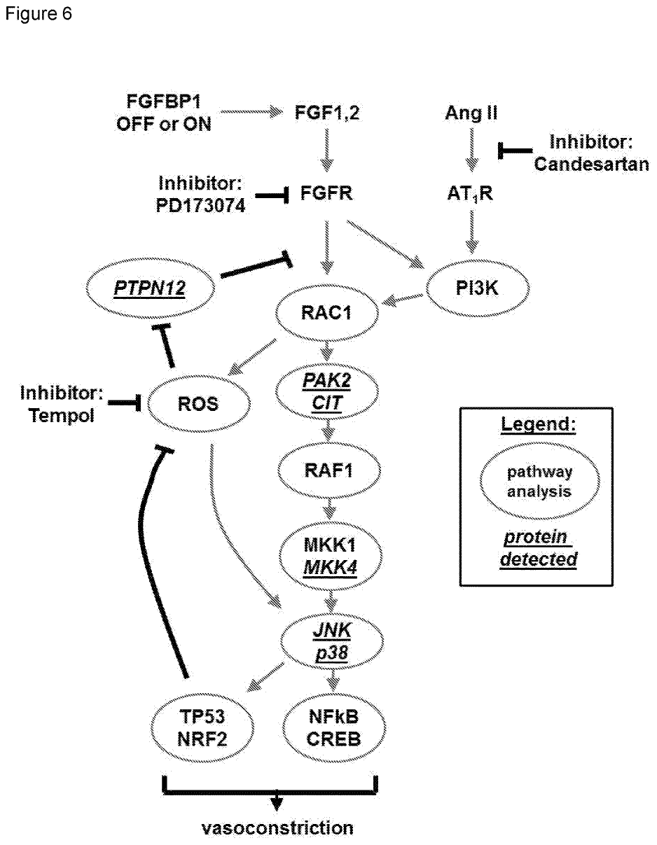

Four proteins that integrate into the known signaling pathway were covered by at least 8 distinct peptides (FIG. 6). Two of these pY-complexed proteins identified were increased by ˜4-fold after BP1 induction and are known downstream effectors of the Rho family of small G-proteins that regulate a multitude of cellular functions, PAK2 (p21 CDC42/RAC1-activated kinase 2) and CIT/STK21 (citron or RHO-interacting, serine/threonine kinase 21). In earlier studies it has been reported that Ras-related small GTP-binding proteins such as RAC1 function as cellular regulators of ROS, an effect that was mimicked by exogenously added growth factors. A third protein, MKK4 (MAP2K4) was found 3.6-fold upregulated in a pY complex after BP1 expression. MKK4 integrates mitogenic and stress signaling and targets the MAP kinases JNK and p38. A fourth protein, PTPN12 (Protein Tyrosine Phosphatase, Non-Receptor Type 12) was found reduced by 2.8-fold after BP1 induction. In general, PTPs are thought to function as negative feed-back controls of tyrosine kinase activities that can be inactivated by ROS signaling. PTPN12 has been linked to altered oxidative stress and its loss to enhanced oncogenic signaling.

For an independent validation of the nodes of signaling uncovered in the above studies, kidney tissues were analyzed by immunohistochemical staining. Staining of parallel tissue sections for phospho-proteins in the MAP kinase pathway downstream of the FGF R1 showed an increase in phospho-MKK4, phospho-p38 and phospho-JNK after BP1 expression (FIG. 5C). For p38, the induction of phosphorylation was confirmed by Western blotting of protein extracts (FIG. 5D).

Cultured kidney cells (HEK293) were used to expand the above findings in an animal model to human cells. Combinations of increasing concentrations of FGF2 and/or AngII were studied for their induction of phosphorylation of MKK4 and the MAP kinases ERK, JNK and p38. An increase in phospho-MKK4, phospho-JNK and phospho-p38 was found in the co-stimulation with FGF2 and AngII. In contrast, co-stimulation with FGF and AngII did not induce phospho-ERK. A parallel analysis in human endothelial and smooth muscle cells corroborates this analysis. After co-stimulation with FGF2 and AngII phospho-p38 was induced in contrast to phospho-JNK and phospho-ERK.

What is claimed is:

1. A method of treating hypertension in a subject in need of treatment thereof, the method comprising administering a pharmaceutically effective amount of an angiotensin II inhibitor and a pharmaceutically effective amount of a receptor tyrosine kinase inhibitor to the subject.

2. The method of claim 1, wherein the angiotensin II inhibitor is an angiotensin converting enzyme (ACE) inhibitor or an angiotensin II receptor antagonist.

3. The method of claim 2, wherein the receptor tyrosine kinase inhibitor is a tyrosine kinase inhibitor, a ligand trap or an antibody specific for the receptor tyrosine kinase.

4. The method of claim 3, wherein the receptor tyrosine kinase inhibitor inhibits the activity of fibroblast growth factor receptor 1 (FGFR1), fibroblast growth factor receptor 2 (FGFR2), fibroblast growth factor receptor 3 (FGFR3) or fibroblast growth factor receptor 4 (FGFR4).

5. The method of any of claims 1-4, wherein the angiotensin II receptor antagonist is selected from the group consisting of Olmesartan, Telmisartan, Losartan, Irbesartan, Valsartan, Candesartan, Eprosartan, Azilsartan, Losartan/hydrochlorothiazide, Amlodipine/valsartan, Telmisartan/hydrochlorothiazide and Valsartan/hydrochlorothiazide.

6. The method of any of claims 1-4, wherein the ACE inhibitor is selected from the group consisting of benazepril, captopril, enalapril fosinopril, Lisinopril, moexipril, perindopril, quinapril, ramipril and trandolapril.

7. The method of any of claims 1-6, wherein the receptor kinase inhibitor is the FGF ligand trap FP-1039 (GSK3052230).

8. The method of any of claims 1-6, wherein the receptor kinase inhibitor is the tyrosine kinase inhibitor PD173074 (CAS No. 219580-11-7), AZD4547 (CAS No. 1035270-39-3), BGJ398 (CAS No. 872511-34-7), AP24534 (CAS No. 943319-70-8), BIBF1120 (CAS No. 656247-17-5), JNJ-42756493 (CAS No. 1346242-81-6), TKI-258 (CAS No. 405169-16-6), PHA-739358 (CAS No. 827318-97-8), BMS-540215 (CAS No. 649735-46-6), TKI-258 dilactic acid (CAS No. 852433-84-2), MK-2461 (CAS No. 917879-39-1), BMS-582664 (CAS No. 649735-63-7), SSR128129E (CAS No. 848318-25-2), PRN1371 (CAS No. 1802929-43-6), PD166866 (CAS No. 192705-79-6), BLU554 (CAS No. 1707289-21-1), S49076 (CAS No. 1265965-22-7), SU5402 (CAS No. 215543-92-3), BLU9931 (CAS No. 1538604-68-0), FIN-2 (CAS No. 1633044-56-0), TKI-258 lactate (CAS No. 915769-50-5), CH5183284 (CAS No. 1265229-25-1) or LY2874455 (CAS No. 1254473-64-7).

9. The method of any of claims 1-6, wherein the receptor tyrosine kinase inhibitor is the antibody GP369, BAY1187982 or MFGR1877S.
